(19) United States
(12) Patent Application Publication
(10) Pub. No.: US 2025/0046396 A1
(43) Pub. Date: Feb. 6, 2025

(54) METHOD, APPARATUS, AND COMPUTER-READABLE MEDIUM FOR OPTIMIZED PARTITIONING FOR ASSEMBLY OF NUCLEIC ACID SEQUENCES

(71) Applicant: TeselaGen Biotechnology Inc., San Francisco, CA (US)

(72) Inventors: Eduardo Abeliuk, Miami, FL (US); Juan Andres Ramirez Neilson, Ñuñoa (CL); Andrés Igor Pérez Manriquez, Las Condes (CL)

(21) Appl. No.: 18/790,662

(22) Filed: Jul. 31, 2024

Related U.S. Application Data
(60) Provisional application No. 63/516,695, filed on Jul. 31, 2023.
(60) Provisional application No. 63/574,407, filed on Apr. 4, 2024.

(51) Int. Cl. G16B 30/20

(52) U.S. Cl. CPC G16B 30/20

(57) ABSTRACT

A method, apparatus, and computer-readable medium for optimized partitioning for assembly of nucleic acid sequences, receiving nucleic acid sequences corresponding to target nucleic acids for assembly and synthesis parameters, querying an inventory database based on the nucleic acid sequences to determine first matching nucleic acid subsequences, the inventory database corresponding to nucleic acid subsequences available in an inventory, identifying second matching nucleic acid subsequences based on one or more overlaps between the nucleic acid sequences, generating an acyclic directed graph data structure corresponding to potential partitions of the nucleic acid sequences based on the first matching nucleic acid subsequences, the second matching nucleic acid subsequences, and one or more synthesis parameters, and determining an optimal partitioning of the nucleic acid sequences based on an optimal path through the acyclic directed graph data structure that minimizes a total weight of traversed edges and nodes within the acyclic directed graph data structure.

### RELATED APPLICATION DATA

This application claims priority to U.S. Provisional Application No. 63/516,695, filed Jul. 31, 2023 and U.S. Provisional Application No. 63/574,407, filed Apr. 4, 2024, the disclosures of which are hereby incorporated by reference in its entirety.

### BACKGROUND

Currently, synthesizing arbitrarily long sequences de novo is impossible and is limited by the error rate introduced by the specific deoxyribonucleic acid (DNA) synthesis technology. Due to limitations of the existing technology, synthesis of large sequences introduces an unacceptable level of errors into the target sequences. Additionally, different synthesis platforms impose differing constraints such as the minimum and maximum allowable DNA length to be synthesized. This represents a problem because there is a need from the industry to synthesize increasingly longer DNA sequences useful for various applications, where sequences longer than 2,000 bp are needed. Furthermore, designing DNA partitioning and assembly strategies for the construction of complex DNA libraries from smaller DNA oligos, synthons or amplicons can be quite cost prohibitive, making it impossible to build libraries covering a range of genetic variations

Accordingly, improvements are needed in technology for partitioning nucleic acid sequences for assembly.

### BRIEF DESCRIPTION OF THE DRAWINGS

FIG. 1 illustrates a flowchart for optimized partitioning for assembly of nucleic acid sequences according to an exemplary embodiment.

FIG. 2 illustrates a system chart for optimized partitioning for assembly of nucleic acid sequences according to an exemplary embodiment.

FIG. 3 illustrates a flowchart for generating an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters according to an exemplary embodiment.

FIG. 4 illustrates an example of determining a first plurality of matching nucleic acid subsequences according to an exemplary embodiment.

FIG. 5 illustrates an example of determining a second plurality of matching nucleic acid subsequences according to an exemplary embodiment.

FIGS. 6A-6C illustrate an example of generating an acyclic directed graph data structure corresponding to potential partitions of the plurality of nucleic acid sequences according to an exemplary embodiment.

FIG. 7 illustrates a flowchart for synthesizing target nucleic acids according to an exemplary embodiment.

FIG. 8 illustrates a hierarchical assembly with 16 assembly pieces according to an exemplary embodiment.

FIG. 9 illustrates the components of a specialized computing environment for optimized partitioning for assembly of nucleic acid sequences according to an exemplary embodiment.

### DETAILED DESCRIPTION

While methods, apparatuses, and computer-readable media are described herein by way of examples and embodiments, those skilled in the art recognize that methods, apparatuses, and computer-readable media for optimized partitioning for assembly of nucleic acid sequences are not limited to the embodiments or drawings described. It should be understood that the drawings and description are not intended to be limited to the particular form disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “can” is used in a permissive sense (i.e., meaning having the potential to) rather than the mandatory sense (i.e., meaning must). Similarly, the words “include,” “including,” and “includes” mean including, but not limited to.

The disclosed methods, apparatuses, and computer-readable media overcome current technical limitations for de novo synthesis of DNA molecules and utilize a novel partitioning algorithm and novel data structures that can take a long DNA sequence and partition it into small elements that can be more easily synthesized or reused from an existing inventory of parts. Thus, for a long sequence of interest (typically sequences between 2,000 bp and 100,000 bp in length), the present system is able to identify segments to be synthesized separately and that can be easily assembled. At the same time, since a laboratory may store samples with different DNA sequences, the present method identifies and utilizes available material parts within inventory, thereby improving efficiency of the DNA synthesis process.

The disclosed methods, apparatuses, and computer-readable media are able to partition sequences in a manner compatible with multiple DNA assembly strategies, including assembly strategies based on flanking homology (e.g., Gibson) and assembly strategies based on type II endonucleases (e.g., Golden Gate). The disclosed system and method can partition either linear or circular DNA sequences. Additionally, the disclosed system and method can be used with optional inventory sequences as input, available in an inventory of existing DNA products.

By querying an inventory database to utilize inventory sequences and reusing those sequences in the design process, the user of the algorithm can radically improve synthesis speeds and efficiency.

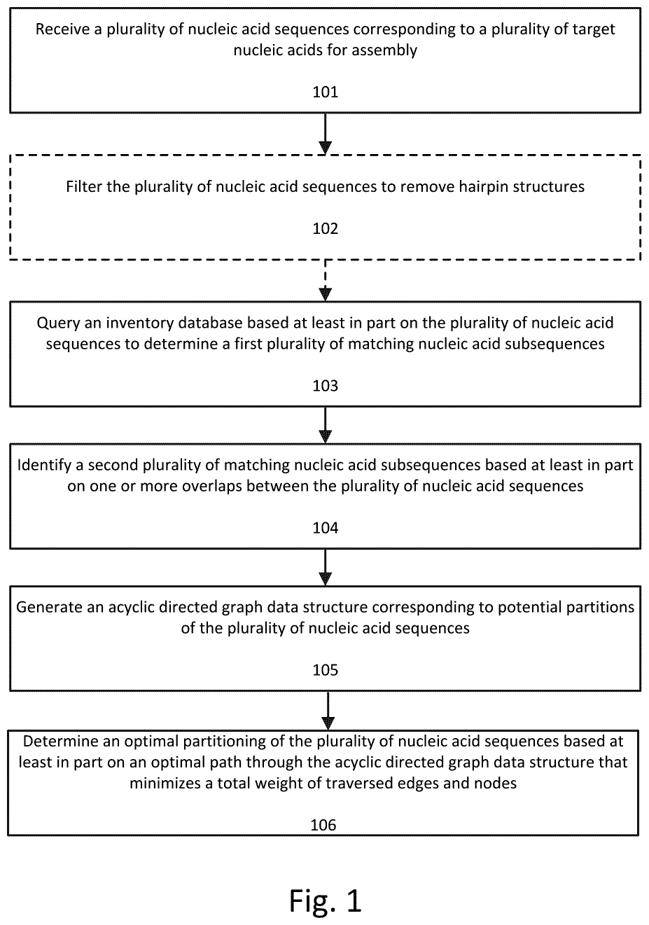

FIG. 1 illustrates a flowchart for optimized partitioning for assembly of nucleic acid sequences according to an exemplary embodiment.

At step 101 one or more or a plurality of nucleic acid sequences corresponding to a plurality of target nucleic acids for assembly and a plurality of synthesis parameters are received. This step can include receiving identifiers corresponding to the base pairs or components of each target nucleic acid.

The system can receive one target sequence or multiple target sequences for assembly. While the present system describes lengthy target DNA sequences for assembly, it is understood that the target sequence can also be a ribonucleic acid (RNA) or an amino acid sequence.

The system can also receive a plurality of synthesis parameters. The synthesis parameters can include one or more of the following parameters:
a subsequence synthesis length range: a range size allowed for synthesis;
an assembly strategy: a type of assembly strategy, which can include Digest based strategies or strategies based in Type-II endonucleases;
one or more inventory partition parameters: one or more parameters for configuring how the inventory is processed and can allow for limits to be established with respect to the size of inventory matches;
a guanine or cytosine content overlap range: a guanine or cytosine content range allowed for overlaps;
a subsequence overlap length: a range of allowed lengths for overlaps;
one or more overlap configuration parameters: these can include several additional pre-implemented rules for overlap validation, which allow determination of the best positions for segment divisions. The available configurable rules can include:
   i. Number of tandem repeats: evaluates overlaps in terms of the number of tandem repeats of base pairs;
   ii. Overlap sequence duplication: helps avoid overlap sequences repeated over the target;
   iii. Homopolymer stretches: helps detect on time overlaps that can generate assembly problems due to homopolymer stretches;
   iv. K-mer repeats: Helps avoiding k-mer stretches on overlap sequences
Enzyme digestion parameters: for enzyme digestion based methods, these parameters allow configuration of the list of allowed enzymes and their characterization;
a maximum match length parameter: polymerase chain reaction (PCR) has inherent length restrictions, dictated by the fidelity of the DNA polymerase utilized in the reaction. The maximum match length parameter is useful because PCR frequently serves as the method for amplifying the assembly piece from a DNA template in inventory. The implementation of this parameter splits an oversized match into multiple shorter matches that are added to the inventory match pool; or
a maximum assembly subsequences per reaction: the maximum number of assembly pieces to be included in any individual assembly reaction.

Optionally, a plurality of inventory sequences can be provided along with the plurality of nucleic acid sequences and the plurality of synthesis parameters. In this case, the plurality of inventory sequences can be used in place of, or in addition to, the inventory database discussed below.

At optional step 102 the plurality of nucleic acid sequences can be filtered to remove problematic structures, such as hairpin structures. Before finding optimal partitions of subsequences, the present method can filter out DNA sequence regions that can be problematic for DNA assembly purposes. Such problematic regions can include for example regions that contain hairpin structures. Different methods can be used to identify and filter out such regions. For example, a filter can be used to filter out specific DNA sequences that are known to contain hairpin structures (e.g. inverted terminal repeats in adeno-associated viruses). Another method to filter out sequences that can contain hairpin regions is to use a computationally efficient algorithm (e.g., a method based on dynamic programming), to identify palindromic regions within the target sequence(s) and filter them out. Of course, additional or alternative computational techniques can be used as part of this step to identify and filter out regions that are likely to contain hairpin structures based on secondary structure simulations.

At step 103 an inventory database is queried based at least in part on the plurality of nucleic acid sequences to determine a first plurality of matching nucleic acid subsequences, wherein the inventory database corresponds to a plurality of nucleic acid subsequences available in an inventory.

In this step, the algorithm compares each target sequence with a database of inventory sequences. It looks for continuous segments where the sequence at the inventory element matches a target. As a result, this step returns a list of inventory matches. Each inventory match contains information about the related inventory sequence and target sequence. The step also returns range (indexes) where the alignment was found on each sequence. There can be multiple inventory matches between one target and one inventory sequence. The sequence can be aligned with the matching inventory sequences.

This step can be implemented by using any sequence alignment algorithm that can find exact sequence matches between sequences. For example, a BLAST (Basic Local Alignment Search Tool) algorithm can be used comparing primary biological sequence information, such as nucleotide sequences of DNA or RNA, or amino acid sequences of proteins. However, BLAST can miss nucleotide matches due to factors like scoring parameters, stringent E-value thresholds, database composition, sequence divergence, algorithm limitations, and suboptimal parameter settings. Therefore, the present system can use BLAST initially for finding matches against the inventory database, as it is very efficient for processing large amounts of data. The detected alignments however, are afterwards analyzed for potential mismatches (missed matching positions at the tails) and fixed on missed match positions.

Additionally, but specifically for assembly methods based on type II endonucleases, an optimized search is utilized. Considering that not all matches will be compatible with the enzyme, the search efficiency is improved by first removing from the inventory database those sequences that don't contain valid recognition sites for the selected enzyme.

The first plurality of matching nucleic acid subsequences can also be determined based at least in part on one or more of the plurality of synthesis parameters. The synthesis parameters can, for example, indicate a maximum or minimum matching length when comparing the subsequences in the inventory to the target sequences.

At step 104 a second plurality of matching nucleic acid subsequences are identified based at least in part on one or more overlaps between the plurality of nucleic acid sequences. In order to improve efficiency by reutilizing synthetic sequences, the present system also searches for matches making pairwise comparisons between all target sequences. This step identifies matches between and within subsequences of the target sequences, eliminating the need to synthesize the same subsequence multiple times.

The second plurality of matching nucleic acid subsequences can also be determined based at least in part on one or more of the plurality of synthesis parameters. The synthesis parameters can, for example, indicate a maximum or minimum matching length when comparing the subsequences in target sequences to other subsequences in target sequences.

The one or more nucleic acid sequences in the plurality of nucleic acid sequences can include circular sequences. In this case, the first plurality of matching nucleic acid subsequences and the second plurality of matching nucleic acid subsequences can be determined based at least in part on one or more rotations of the circular sequences. This ensures that matches are not missed to the particular alignment of a circular sequence. As a way to avoid discarding promising matches (based on the inventory or matching subsequences) that cross the origin of the circular sequences, this process can identify positions where the target sequences can be rotated to prevent dismissing useful matches and have the most possible coverage from reusable sequences.

One or more optimization steps can be performed to reduce the number of matching subsequences in the first plurality of matching nucleic acid subsequences and the second plurality of matching nucleic acid subsequences. In particular, redundant matches can be removed to improve efficiency and save resources. Matches that are removed can be replaced with a reference to a copy of the corresponding subsequence.

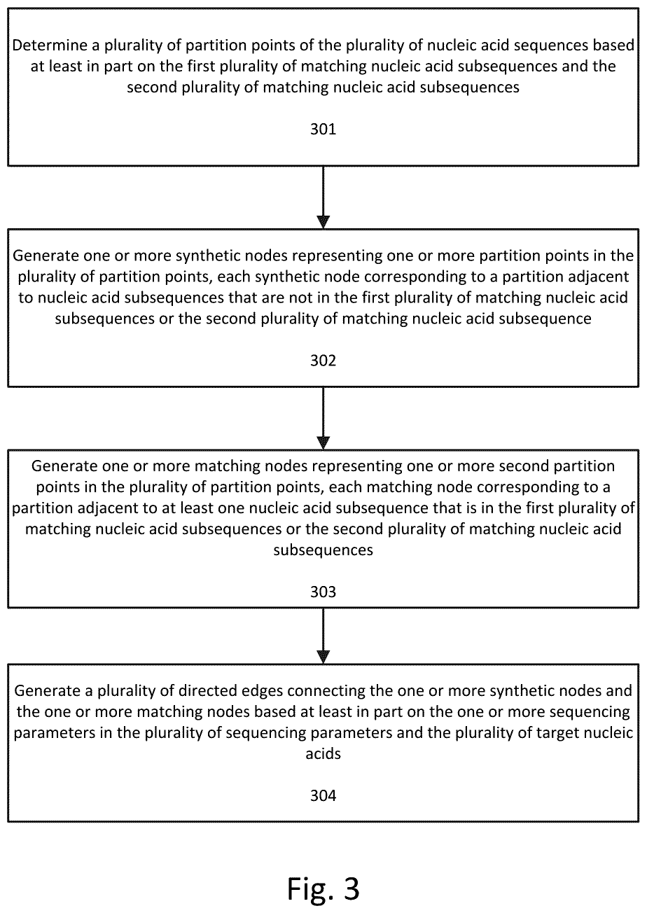

At step 105 an acyclic directed graph data structure is generated corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters. This step is explained in greater detail with respect to FIG. 3, described below. Optionally, an acyclic directed graph data structure can be generated for each target nucleic acid sequence in the plurality of target nucleic acid sequences.

Prior to generating the acyclic directed graph structure, a filter step can also be performed to filter out one or more partitions or subsequences and reduce the overall size of the directed graph that is created. In this filtering step the non-matching sequences can be divided into ranges or tiers of cost and only sequences having lower associated costs (determined based on synthesis parameters) can be utilized for the graph.

At step 106 of FIG. 1 an optimal partitioning of the plurality of nucleic acid sequences is determined based at least in part on an optimal path through the acyclic directed graph data structure that minimizes a total weight of traversed edges and nodes within the acyclic directed graph data structure.

The optimization step includes the process of solving the optimization problem that is encoded in the acyclic directed graph data structure. The optimization problem can be solved by determining the lowest weight or lowest cost path through the acyclic directed graph data structure. The solution can be determined using an optimization algorithm, such as the Dijkstra algorithm, or another algorithm for finding the shortest path through a weighted graph.

When an acyclic directed graph data structure is generated for each target nucleic acid sequence, the optimization step can be parallelized and run individually for each target sequence's graph. In this case, the optimization step will produce a solution for each target sequence.

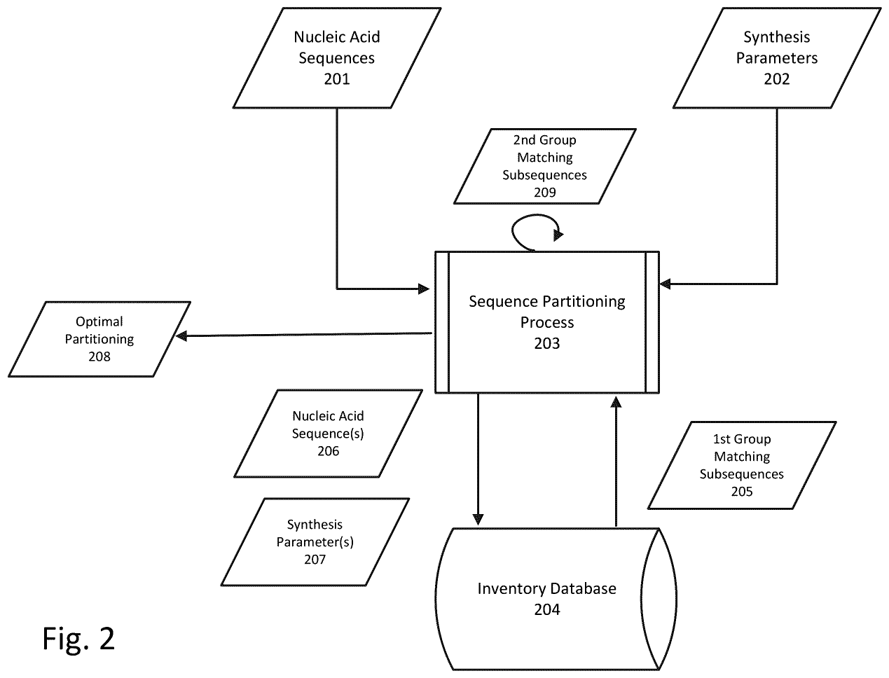

FIG. 2 illustrates a system chart for optimized partitioning for assembly of nucleic acid sequences according to an exemplary embodiment.

As shown in FIG. 2, nucleic acid sequences 201 (corresponding to target sequences) and synthesis parameters 202 are provided to sequence partitioning process 203. The sequence partitioning process 203 queries the inventory database 204 with one or more nucleic acid sequences 206 and (optionally) one or more of the synthesis parameters 207 and determines the first plurality of matching nucleic acid subsequences 205. Additionally, the sequence partitioning process 203 compares subsequences within and across the target sequences and determines a second plurality of matching nucleic acid subsequences 209. The sequence partitioning process 203 then generates an acyclic directed graph data structure corresponding to potential partitions of the plurality of nucleic acid sequences and determines an optimal partitioning 208 of the target sequences.

FIG. 3 illustrates a flowchart for generating an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters according to an exemplary embodiment.

At step 301 a plurality of partition points of the plurality of nucleic acid sequences are determined based at least in part on the first plurality of matching nucleic acid subsequences and the second plurality of matching nucleic acid subsequences.

This step can include identifying matching nucleic acid subsequences within the plurality of nucleic acid sequences (target sequences) that match any of the the first plurality of matching nucleic acid subsequences or the second plurality of matching nucleic acid subsequences. Once the matches are identified, the position prior to the start of each of these matching subsequences can be designated as partition point. Additionally, the position subsequent to the end of each of the matching subsequences can be designated as partition point.

One or more partition points in the plurality of partition points can also be determined based at least in part on one or more synthesis parameters in the plurality of synthesis parameters. The synthesis parameters can be used in conjunction with the matching nucleic acid subsequences. For example, the synthesis parameters can indicate that only matching nucleic acid subsequences having a minimum length should be utilized, in which case the system will disregard matching nucleic acid subsequences have a length less than the minimum length when determining partition points.

One or more partition points can also be determined based solely on one or more synthesis parameters in the plurality of synthesis parameters. For example, the synthesis parameters can specify a desired length of subsequences or a synthesis methodology that results in one or more desirable partitions of subsequences. In another example, certain subsequences can be flagged as desirable from a synthesis perspective for a particular methodology, and any time those subsequences occur, the positions prior to and after those sequences can be designated as partition points.

Steps 302 and 303 involve transforming some of the partitioning parameters and matches (inventory as well as targets) into nodes. A node corresponds to a candidate partitioning position around where the sequence will be broken into parts. As explained below, the system generates two types of nodes: synthetic nodes and matched nodes.

At step 302 one or more synthetic nodes representing one or more partition points in the plurality of partition points are generated, each synthetic node corresponding to a partition adjacent to nucleic acid subsequences that are not in the first plurality of matching nucleic acid subsequences or the second plurality of matching nucleic acid subsequences. In other words, synthetic nodes correspond to partitions that are not adjacent to any subsequence that is found in the first plurality of matching nucleic acid subsequences or the second plurality of matching nucleic acid subsequences.

Synthetic nodes are nodes that, if used for the final partitioning, each of the connected parts (subsequences) are required to be synthetic (i.e., not in the inventory database and not found in other subsequences of the target sequences). Every base pair position of each target sequence can be considered a synthetic node, but some of these can be discarded in the process based on validation rules regarding the suitability of the partition position for a specific assembly method.

At step 303 one or more matching nodes representing one or more second partition points in the plurality of partition points are generated, each matching node corresponding to a partition adjacent to at least one nucleic acid subsequence that is in the first plurality of matching nucleic acid subsequences or the second plurality of matching nucleic acid subsequences.

Matching nodes are nodes corresponding to partitions where at least one of the neighboring parts (subsequences) matches a subsequence in the inventory database or matches a subsequence found in one or more of the target subsequences. This is a condition for just one of the two neighboring segments. The other can be a synthetic subsequence or another matching part. Matching nodes are created based on alignment results from the process used to find matches between target sequences.

Steps 301-303 can be customized according to different target sequence assembly methods and information defining those sequence assembly methods can be passed into the method using the synthesis parameters. These parameters can be used to determine a score or weighting to assign to each node. For example, a synthesis technique which is time consuming or resource intensive can result in a synthetic node having a higher relative weighting, whereas a simple synthesis technique can result in a synthetic node having a lower relative weighting. Similarly, a simple process of generating a subsequence from inventory can result in a matched node having a lower relative weighting. The inner properties (including weightings or scores) of nodes will depend on the selected assembly (e.g., synthesis, extraction, etc.) strategy. This allows the system to encode different characteristics of different assembly methods. These scores or weightings are utilized when determining an optimal set of partitions, as will be described further below.

Additionally, is important to note that there can be more than one node per position, as nodes can have different qualities. For example, there can be both synthetic and matched nodes for a particular partition position.

At step 304 a plurality of directed edges connecting the one or more synthetic nodes and the one or more matching nodes are generated based at least in part on the one or more synthesis parameters in the plurality of synthesis parameters and the plurality of target nucleic acids.

Directed edges are generated corresponding to directed paths through the graph (from start to end) that result in the desired plurality of nucleic acid sequences (i.e., the target sequences for assembly).

As described above, nodes correspond to partition position (i.e., cut position) candidates. The present system encodes the subsequences of the target sequences in a specialized data structure in the form of a specialized directed graph, such that the subsequences that minimize synthesis costs/overhead and maximize the probability of successful assembly can be determined by optimizing a path through the directed graph data structure. Using the specialized directed graph, this optimization problem is a routing problem, where the objective is to minimize the overall costs of traveling along the defined nodes from the starting point of the sequence to its end.

In other words, the problem is modeled as an acyclic directed graph where the optimizer must search the best route from the beginning node (sequence's start) to the end node (sequence's end), minimizing the costs of traveling through the edges. The current step involves the creation of the edges in the mentioned graph, where the optimization function and the problem's restrictions (provided via synthesis parameters) are translated into, and represented in the form of edge properties (existence of link and associated cost/weightings) between nodes.

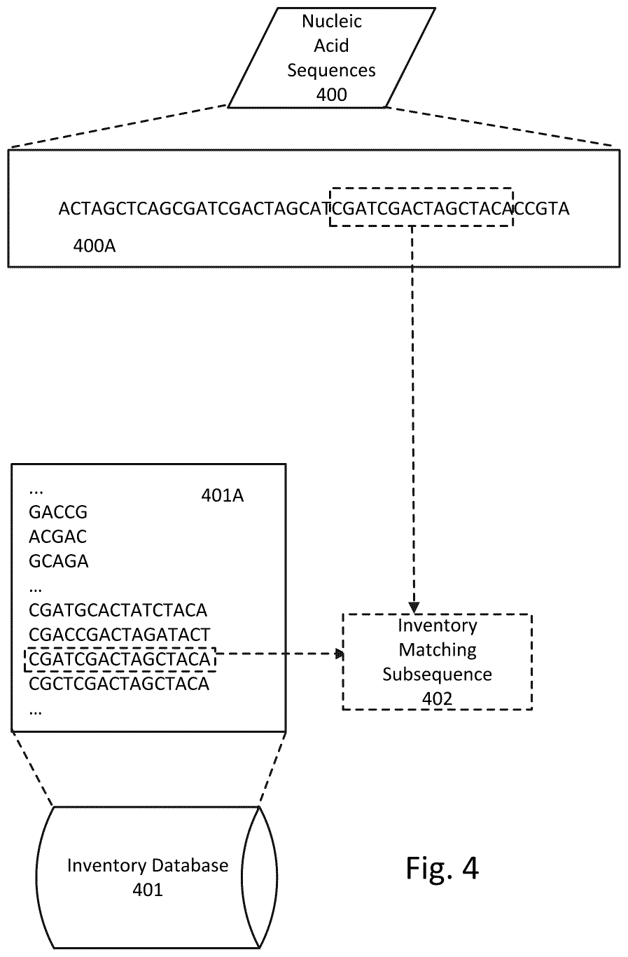

An example of the above-described steps will now be explained with respect to FIGS. 4, 5, and 6A-6C. FIG. 4 illustrates an example of determining a first plurality of matching nucleic acid subsequences according to an exemplary embodiment. As shown in FIG. 4, a nucleic acid sequence 400A in the plurality of nucleic acid sequences 400 is compared with a plurality of inventory subsequences 401A in the inventory database 401 to identify an inventory matching subsequence 402. The example shown in FIG. 4 illustrates a single target sequence 400A for simplicity, but it is understood that the subsequences of the inventory can be compared with multiple target sequences.

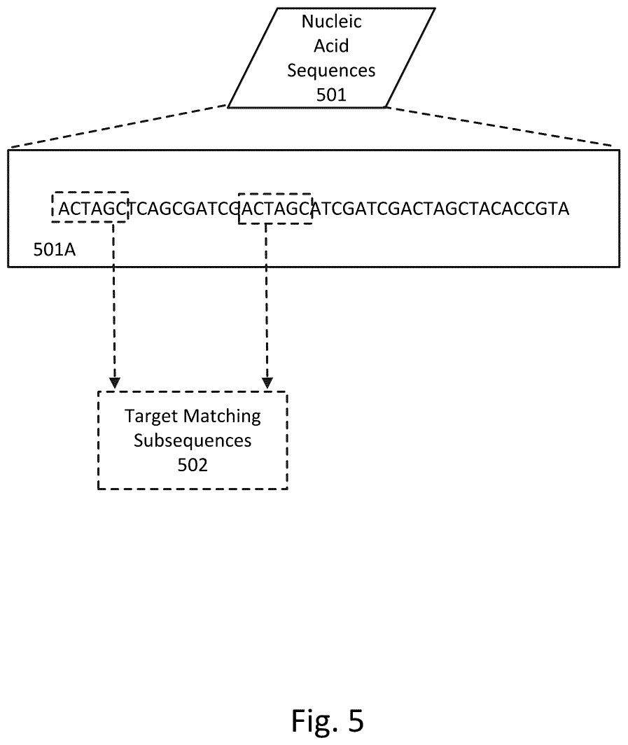

FIG. 5 illustrates an example of determining a second plurality of matching nucleic acid subsequences according to an exemplary embodiment. The example shown in FIG. 5 illustrating matching subsequences within a single target sequence for simplicity, but it is understood that matching subsequences can be identified across multiple target sequences. As shown in FIG. 5, matching subsequences are identified 502 within a nucleic acid sequence 501A in the plurality of nucleic acid sequences 501.

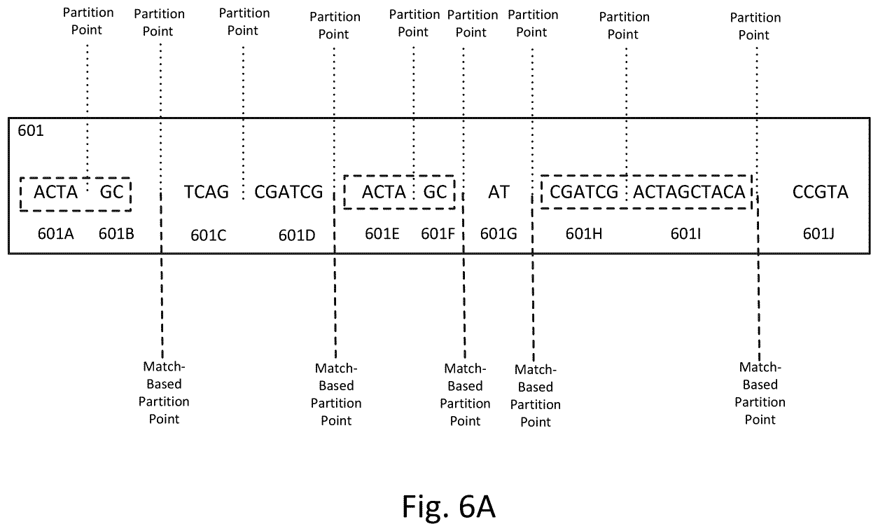

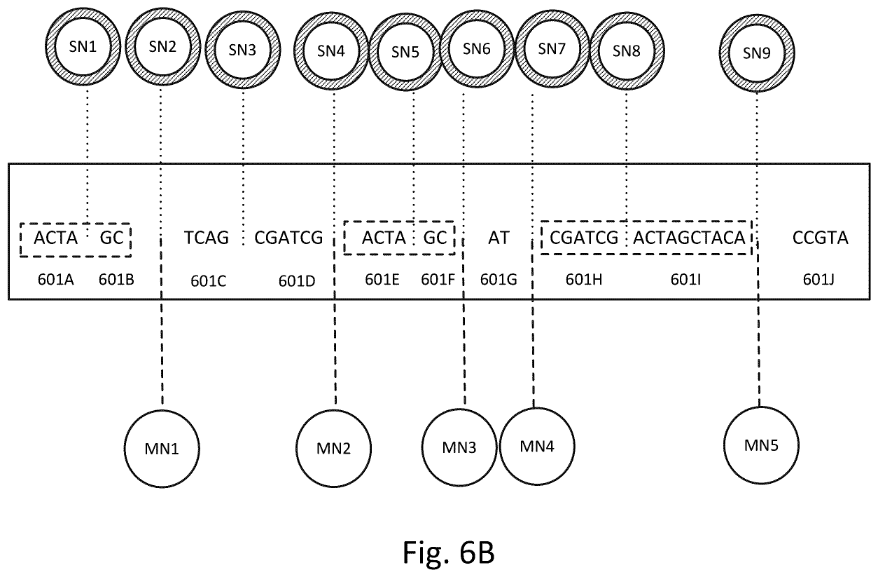

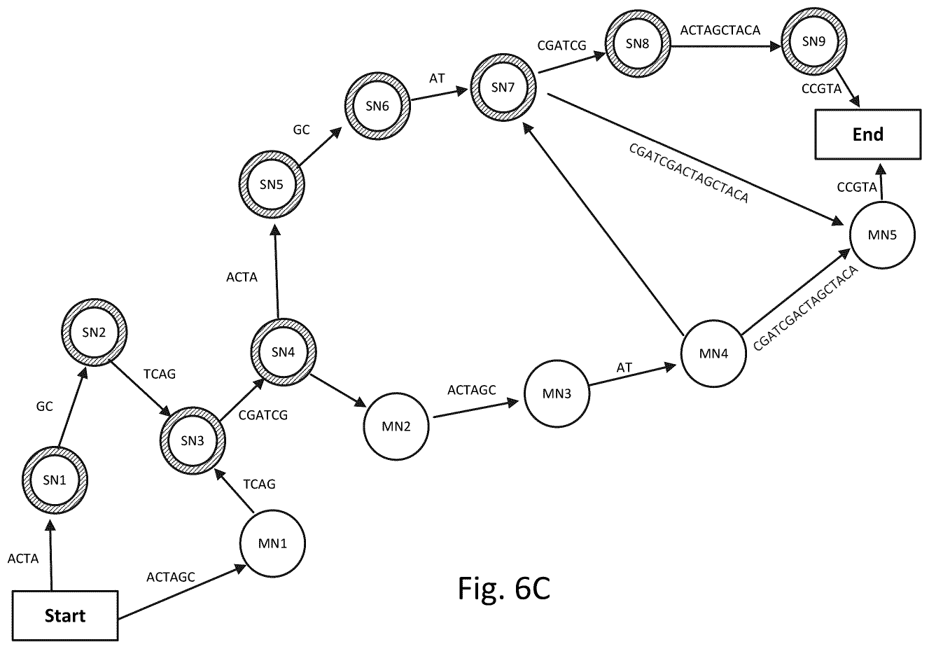

FIGS. 6A-6C illustrate an example of generating an acyclic directed graph data structure corresponding to potential partitions of the plurality of nucleic acid sequences according to an exemplary embodiment. For the purpose of clarity, the example shown in FIGS. 6A-6C illustrates the generation of acyclic directed graph data structure for a single nucleic acid sequence 601 in the plurality of nucleic acid sequences. Of course, it is understood that the described method can be used to generate an acyclic directed graph data structure corresponding to multiple different nucleic acid sequences.

FIG. 6A illustrates a single nucleic acid sequence 601 divided into a plurality of subsequences. The dashed rectangles corresponding to subsequences 601A and 601B and subsequences 601E and 601F indicate an overlapping (matching) subsequence within the nucleic acid sequence 601 (see FIG. 5). Additionally, the dashed rectangle corresponding to subsequences 601H and 601I indicates a subsequence that was found (matched) in the inventory database (see FIG. 4).

The dashed lines in FIG. 6A indicate “match-based partition points” in the nucleic acid sequence 601. As shown in FIG. 6A, there are five match-based partition points in the nucleic acid sequence. The match-based partition points are designated as the positions before and after subsequences that match either subsequences in the inventory database or other subsequences in the nucleic acid sequence (or other nucleic acid sequences if more than one). As shown in FIG. 6A, match-based partition points are designated after the subsequence indicated by the first dashed rectangle, before and after the subsequence indicated by the second dashed rectangle, and before and after the subsequence indicated by the third dashed rectangle.

The dotted lines in FIG. 6A indicated additional partition points in the nucleic acid sequence 601. These partition points can be determined based one or more factors, such as one or more synthesis parameters, an assembly method, specific characteristics of the nucleic sequences, minimum or maximum length parameters, or other values. As shown in FIG. 6A, there are nine partition points in the nucleic acid sequence.

FIG. 6B illustrates the process of generating the nodes, including synthetic nodes and matching nodes. As shown in FIG. 6B, matching nodes are generated corresponding to the match-based partition points and synthetic nodes are generated corresponding to the other partition points (i.e., the non-match-based partition points).

FIG. 6C illustrates an acyclic directed graph data structure according to an exemplary embodiment. As shown in FIG. 6C, the graph includes the synthetic nodes and matching nodes, as well as directed edges between the nodes. As shown in the graph, every path through the directed graph from the start point to the end point results in creation of the nucleic acid sequence. The directed edges can connect synthetic nodes, matching nodes, or both synthetic and matching nodes. As shown in FIG. 6C, some of the directed edges can correspond to subsequences, but other directed edges do not necessarily correspond to subsequences (such as the edge between MN4 and SN7).

The nodes and directed edges in the graph can be assigned different weights/costs depending on the corresponding subsequence, the production method of the corresponding subsequence, and other synthesis parameters. For example, a matching node or an edge to matching node can have a lower weight than a synthetic node or an edge to a synthetic node to indicate that the subsequence corresponding to the matching node can be more efficiently produced than the subsequence corresponding to the synthetic node. In another example, matching nodes or related edges that correspond to subsequences in the inventory database can have a different weight than matching nodes or related edges that correspond to matching subsequences in the target sequences.

Each edge in the plurality of directed edges can have one or more associated edge properties and the one or more associated edge properties can be determined based at least in part on one or more of whether the edge connects to a synthetic node or a matching node or the one or more synthesis parameters in the plurality of synthesis parameters.

To determine an optimal partitioning of the nucleic acid sequence, the optimal path through the directed graph in FIG. 6C can be determined, taking in account the weightings and properties of the nodes and edges within the data structure.

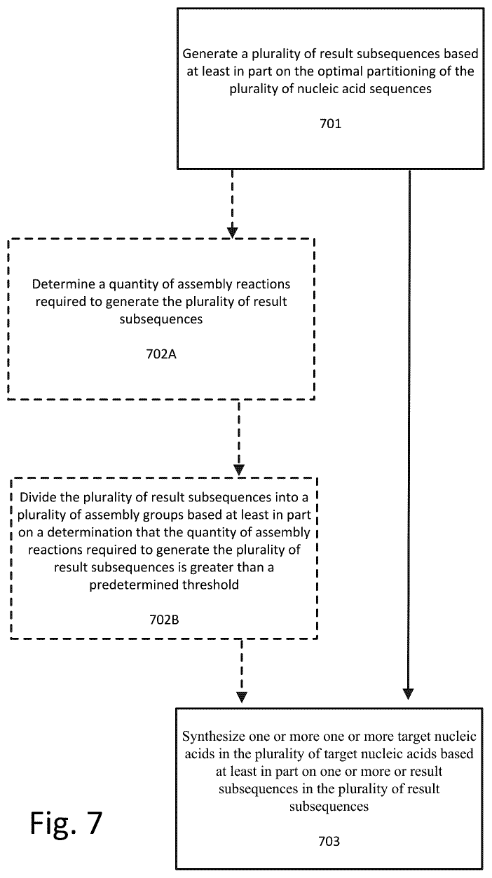

FIG. 7 illustrates a flowchart for synthesizing target nucleic acids according to an exemplary embodiment.

At step 701 a plurality of result subsequences are generated based at least in part on the optimal partitioning of the plurality of nucleic acid sequences, each result subsequence indicating a corresponding source. The one or more target nucleic acids in the plurality of target nucleic acids are configured to be sequenced based at least in part on one or more or result subsequences in the plurality of result subsequences.

The resulting node paths obtained for each target from the optimization process can be translated into a segmented sequence, where each part is delimited by 2 optimal nodes from the path. The output contains information corresponding to the calculated partition parts. For each of these partition parts, the output can include a complete sequence, indexes (start, end) at the target, and information about its source (synthetic, target matching synthetic, or inventory). Non-synthetic portions can include indexes from the source in the inventory. Additionally, digest portions can contain information about the enzyme and digested fragment. PCR portions can include the proposed overlaps that can be used to assemble the subsequences.

The resulting node paths and the output can be transmitted on a user interface, for example, as a visualization showing the base-pairs, order of sequences, and partitions.

At step 703 one or more one or more target nucleic acids in the plurality of target nucleic acids are synthesized based at least in part on one or more or result subsequences in the plurality of result subsequences. In the case of inventory subsequences, they can be extracted from DNA samples available in inventory (either by PCR or enzymatic digestion).

When the length of the DNA sequence to be partitioned is too large, and the number of assembly pieces exceeds a certain optimal number, the assembly reaction might be unreliable and error prone. Therefore, the present system includes controls to limit the total number of assembly pieces in each assembly reaction to a specific number (which might range from, say, 2 to 15). These are described with respect to FIGS. 7A-7B.

At optional step 702A a quantity of assembly reactions required to generate the plurality of result subsequences are determined.

At optional step 702B the plurality of result subsequences are divided into a plurality of assembly groups based at least in part on a determination that the quantity of assembly reactions required to generate the plurality of result subsequences is greater than a predetermined threshold.

For this procedure, the system can incorporate an additional input:

max_num_assembly_pieces_per_reaction: Maximum number of assembly pieces to be included in any individual assembly reaction.

The algorithm can proceed as already described above, by calculating the number of nodes and therefore the number of assembly pieces needed. This value can stored as:

$$total\_num \_of \_assembly \_pieces ( = number of nodes + 1 ) .$$

The number of assembly reactions needed in the level 0 hierarchical assembly step can be determined as: num_level_0_assembly_reactions=

ceiling(total_num_of_assembly_pieces/max_num_assembly_pieces_per_reaction)

If num_level_0_assembly_reactions>1 and num_level_0_assembly_reactions<=max_num_assembly_pieces_per_reaction then the assembly pieces (and nodes) can be split into num_level_0_assembly_reactions subsets of assembly pieces that can then be individually assembled by following steps 5 through 8 of our algorithm.

These individual assembly reactions can then be further assembled into a final assembly reaction. If num_level_0_assembly_reactions>max_num_assembly_pieces_per_reaction,

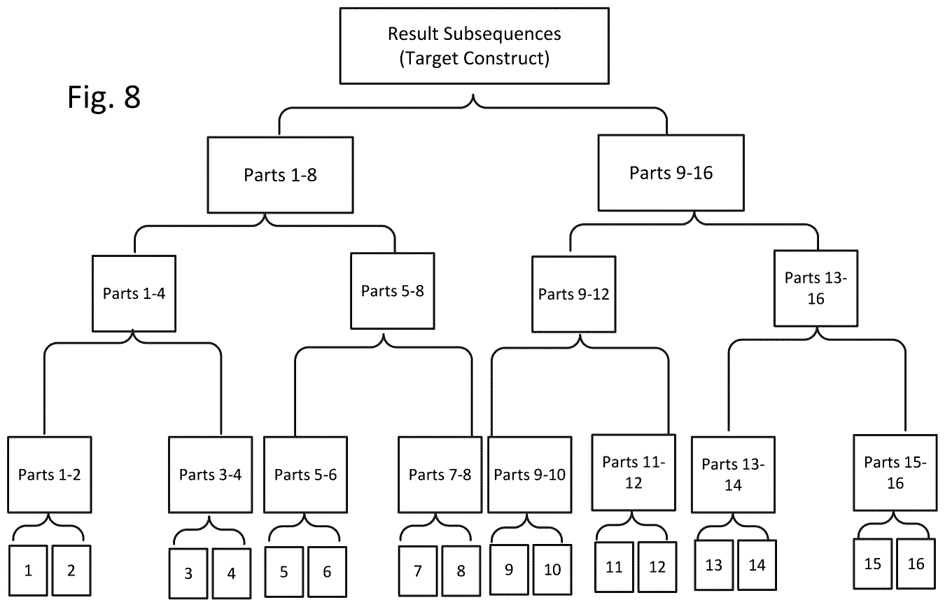

The assembly pieces can then be organized into a hierarchical assembly tree. FIG. 8 illustrates a hierarchical assembly with 16 assembly pieces according to an exemplary embodiment.

As shown in FIG. 16, total_num_of_assembly_pieces=16 and max_num_assembly_pieces_per_reaction=2.

In this case, num_level_0_assembly_reactions=ceiling(16/2)=8. This generates a new level as 8>2

$$num\_level \_ 1 \_assembly \_reactions = ceiling ( 8 / 2 ) = 4$$

$$num\_level \_ 2 \_assembly \_reactions = ceiling ( 4 / 2 ) = 2$$

$$num\_level \_ 3 \_assembly \_reactions = ceiling ( 2 / 2 ) = 1$$

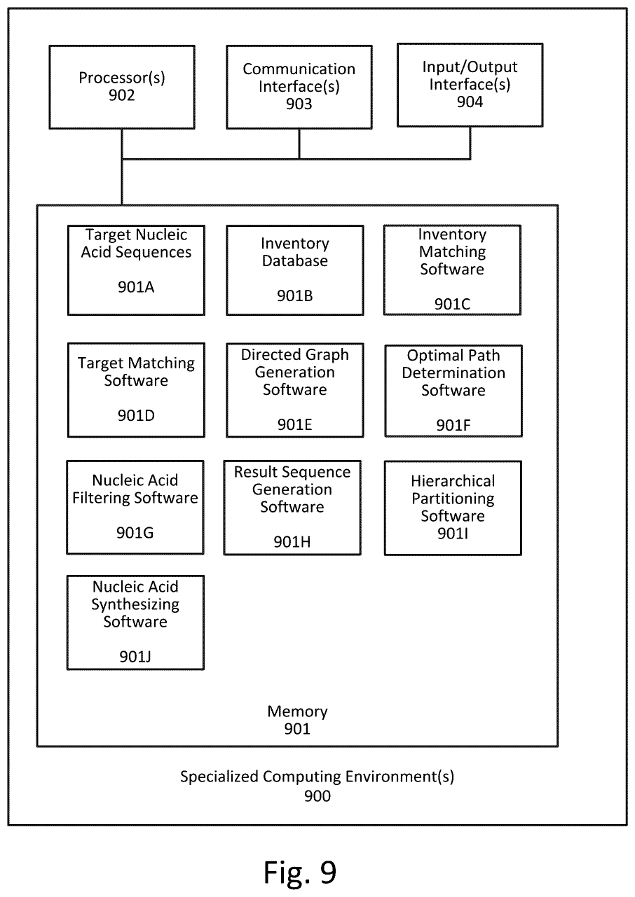

FIG. 9 illustrates the components of a specialized computing environment for optimized partitioning for assembly of nucleic acid sequences according to an exemplary embodiment. Specialized computing environment 900 can be made up of one or more computing devices that include a memory 901 that is a non-transitory computer-readable medium and can be volatile memory (e.g., registers, cache, RAM), non-volatile memory (e.g., ROM, EEPROM, flash memory, etc.), or some combination of the two.

As shown in FIG. 9, memory 901 stores target nucleic acid sequences 901A, inventory database 901B, inventory matching software 901C, target matching software 901D, directed graph generation software 901E, optimal path determination software 901F, nucleic acid filtering software 901G, result sequence generation software 901H, hierarchical partitioning software 901I, and/or nucleic acid synthesizing software 901J.

Each of the software components in memory 901 store specialized instructions and data structures configured to perform the methods for optimized partitioning for assembly of nucleic acid sequences described herein.

All of the software stored within memory 901 can be stored as a computer-readable instructions, that when executed by one or more processors 902, cause the processors to perform the functionality described with respect to FIGS. 1-8.

Processor(s) 902 execute computer-executable instructions and can be a real or virtual processors. In a multi-processing system, multiple processors or multicore processors can be used to execute computer-executable instructions to increase processing power and/or to execute certain software in parallel. As discussed earlier in the application, processors can be processors specialized for the task of training and applying a predictive model, such as graphical processing units (GPUs).

Computing environment 900 additionally includes a communication interface 903, such as a network interface, which is used to communicate with devices, applications, or processes on a computer network or computing system, collect data from devices on a network, and implement encryption/decryption actions on network communications within the computer network or on data stored in databases of the computer network. The communication interface conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Computing environment 900 further includes input and output interfaces 904 that allow users (such as system administrators) to provide input to the controller to cause the neurological screening device to display information, to edit data stored in memory 901, or to perform other administrative functions. For example, an administrator can configure, add, or edit, for example, constraints, encoding software, or experimental data points stored in memory 901.

An interconnection mechanism (shown as a solid line in FIG. 9), such as a bus, controller, or network interconnects the components of the computing environment 900.

Input and output interfaces 904 can be coupled to input and output devices. For example, Universal Serial Bus (USB) ports can allow for the connection of a keyboard, mouse, pen, trackball, touch screen, or game controller, a voice input device, a scanning device, a digital camera, remote control, or another device that provides input to the computing environment.

The computing environment 900 can additionally utilize a removable or non-removable storage, such as magnetic disks, magnetic tapes or cassettes, CD-ROMs, CD-RWs, DVDs, USB drives, or any other medium which can be used to store information and which can be accessed within the computing environment 900.

Computing environment 900 can be a set-top box, personal computer, or one or more servers, for example a farm of networked servers, a clustered server environment, or a cloud network of computing devices.

The described system and method has many benefits. It divides long target sequence strings into shorter sequences that can be synthesizable by most DNA providers. The parts are designed so they maximize the probability of assembly during the DNA assembly process for building the final molecules. The present method works for several types of assembly strategies, including methods based in Type-II Endonucleases and flanking homology. The present method supports linear and circular sequences. The present method searches for sequence fragments in an inventory database. These fragments can be used in the construction of the molecule, reducing the costs and overhead and improving efficiency of the synthesizing process.

It will be appreciated by those skilled in the art that changes could be made to the embodiments described above without departing from the broad inventive concept thereof. For example, the steps or order of operation of one of the above-described methods could be rearranged or occur in a different series, as understood by those skilled in the art. It is understood, therefore, that this disclosure is not limited to the particular embodiments disclosed, but it is intended to cover modifications within the spirit and scope of the following claims.

1. A method executed by one or more computing devices for optimized partitioning for assembly of nucleic acid sequences, the method comprising:
receiving, by at least one of the one or more computing devices, a plurality of nucleic acid sequences corresponding to a plurality of target nucleic acids for assembly and a plurality of synthesis parameters;
querying, by at least one of the one or more computing devices, an inventory database based at least in part on the plurality of nucleic acid sequences to determine a first plurality of matching nucleic acid subsequences, wherein the inventory database corresponds to a plurality of nucleic acid subsequences available in an inventory;
identifying, by at least one of the one or more computing devices, a second plurality of matching nucleic acid subsequences based at least in part on one or more overlaps between the plurality of nucleic acid sequences;
generating, by at least one of the one or more computing devices, an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters; and
determining, by at least one of the one or more computing devices, an optimal partitioning of the plurality of nucleic acid sequences based at least in part on an optimal path through the acyclic directed graph data structure that minimizes a total weight of traversed edges and nodes within the acyclic directed graph data structure.

2. The method of claim 1, wherein generating an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters comprises:
determining a plurality of partition points of the plurality of nucleic acid sequences based at least in part on the first plurality of matching nucleic acid subsequences and the second plurality of matching nucleic acid subsequences;
generating one or more synthetic nodes representing one or more partition points in the plurality of partition points, each synthetic node corresponding to a partition adjacent to nucleic acid subsequences that are not in the first plurality of matching nucleic acid subsequences or the second plurality of matching nucleic acid subsequences;
generating one or more matching nodes representing one or more second partition points in the plurality of partition points, each matching node corresponding to a partition adjacent to at least one nucleic acid subsequence that is in the first plurality of matching nucleic acid subsequences or the second plurality of matching nucleic acid subsequences; and
generating a plurality of directed edges connecting the one or more synthetic nodes and the one or more matching nodes based at least in part on the one or more synthesis parameters in the plurality of synthesis parameters and the plurality of target nucleic acids.

3. The method of claim 2, wherein each edge in the plurality of directed edges has one or more associated edge properties, the one or more associated edge properties being determined based at least in part on one or more of:
whether the edge connects to a synthetic node or a matching node; or
the one or more synthesis parameters in the plurality of synthesis parameters.

4. The method of claim 1, wherein the plurality of synthesis parameters comprise one or more of a subsequence synthesis length range, an assembly strategy, one or more inventory partition parameters, a guanine or cytosine content overlap range, a subsequence overlap length, one or more overlap configuration parameters, one or more enzyme digestion parameters, a maximum match length parameter, or a maximum assembly subsequences per reaction.

5. The method of claim 1, wherein:
the first plurality of matching nucleic acid subsequences are determined based at least in part on one or more of the plurality of synthesis parameters; and
the second plurality of matching subsequences are determined based at least in part on one or more of the plurality of synthesis parameters.

6. The method of claim 1, wherein one or more nucleic acid sequences in the plurality of nucleic acid sequences comprise circular sequences and wherein first plurality of matching nucleic acid subsequences and the second plurality of matching subsequences are determined based at least in part on one or more rotations of the circular sequences.

7. The method of claim 1, further comprising:
generating, by at least one of the one or more computing devices, a plurality of result subsequences based at least in part on the optimal partitioning of the plurality of nucleic acid sequences, each result subsequence indicating a corresponding source, wherein one or more target nucleic acids in the plurality of target nucleic acids are configured to be sequenced based at least in part on one or more or result subsequences in the plurality of result subsequences.

8. The method of claim 7, further comprising:
synthesizing, by at least one of the one or more computing devices, one or more one or more target nucleic acids in the plurality of target nucleic acids based at least in part on one or more or result subsequences in the plurality of result subsequences.

9. The method of claim 7, further comprising:
determining, by at least one of the one or more computing devices, a quantity of assembly reactions required to generate the plurality of result subsequences; and
dividing, by at least one of the one or more computing devices, the plurality of result subsequences into a plurality of assembly groups based at least in part on a determination that the quantity of assembly reactions required to generate the plurality of result subsequences is greater than a predetermined threshold.

10. The method of claim 1, further comprising:
filtering, by at least one of the one or more computing devices, the plurality of nucleic acid sequences to remove hairpin structures.

11. An apparatus for optimized partitioning for assembly of nucleic acid sequences, the apparatus comprising:
one or more processors; and
one or more memories operatively coupled to at least one of the one or more processors and having instructions stored thereon that, when executed by at least one of the one or more processors, cause at least one of the one or more processors to:
receive a plurality of nucleic acid sequences corresponding to a plurality of target nucleic acids for assembly and a plurality of synthesis parameters;
query an inventory database based at least in part on the plurality of nucleic acid sequences to determine a first plurality of matching nucleic acid subsequences, wherein the inventory database corresponds to a plurality of nucleic acid subsequences available in an inventory;
identify a second plurality of matching nucleic acid subsequences based at least in part on one or more overlaps between the plurality of nucleic acid sequences;
generate an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters; and
determine an optimal partitioning of the plurality of nucleic acid sequences based at least in part on an optimal path through the acyclic directed graph data structure that minimizes a total weight of traversed edges and nodes within the acyclic directed graph data structure.

12. The apparatus of claim 11, wherein the instructions that, when executed by at least one of the one or more processors, cause at least one of the one or more processors to generate an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters further cause at least one of the one or more processors to:
determine a plurality of partition points of the plurality of nucleic acid sequences based at least in part on the first plurality of matching nucleic acid subsequences and the second plurality of matching nucleic acid subsequences;
generate one or more synthetic nodes representing one or more partition points in the plurality of partition points, each synthetic node corresponding to a partition adjacent to nucleic acid subsequences that are not in the first plurality of matching nucleic acid subsequences or the second plurality of matching nucleic acid subsequences;
generate one or more matching nodes representing one or more second partition points in the plurality of partition points, each matching node corresponding to a partition adjacent to at least one nucleic acid subsequence that is in the first plurality of matching nucleic acid subsequences or the second plurality of matching nucleic acid subsequences; and
generate a plurality of directed edges connecting the one or more synthetic nodes and the one or more matching nodes based at least in part on the one or more synthesis parameters in the plurality of synthesis parameters and the plurality of target nucleic acids.

13. The apparatus of claim 12, wherein each edge in the plurality of directed edges has one or more associated edge properties, the one or more associated edge properties being determined based at least in part on one or more of:
whether the edge connects to a synthetic node or a matching node; or
the one or more synthesis parameters in the plurality of synthesis parameters.

14. The apparatus of claim 11, wherein the plurality of synthesis parameters comprise one or more of a subsequence synthesis length range, an assembly strategy, one or more inventory partition parameters, a guanine or cytosine content overlap range, a subsequence overlap length, one or more overlap configuration parameters, one or more enzyme digestion parameters, a maximum match length parameter, or a maximum assembly subsequences per reaction.

15. The apparatus of claim 11, wherein:
the first plurality of matching nucleic acid subsequences are determined based at least in part on one or more of the plurality of synthesis parameters; and
the second plurality of matching subsequences are determined based at least in part on one or more of the plurality of synthesis parameters.

16. The apparatus of claim 11, wherein one or more nucleic acid sequences in the plurality of nucleic acid sequences comprise circular sequences and wherein first plurality of matching nucleic acid subsequences and the second plurality of matching subsequences are determined based at least in part on one or more rotations of the circular sequences.

17. The apparatus of claim 11, wherein at least one of the one or more memories has further instructions stored thereon that, when executed by at least one of the one or more processors, cause at least one of the one or more processors to:
generate a plurality of result subsequences based at least in part on the optimal partitioning of the plurality of nucleic acid sequences, each result subsequence indicating a corresponding source, wherein one or more target nucleic acids in the plurality of target nucleic acids are configured to be sequenced based at least in part on one or more or result subsequences in the plurality of result subsequences.

18. The apparatus of claim 17, wherein at least one of the one or more memories has further instructions stored thereon that, when executed by at least one of the one or more processors, cause at least one of the one or more processors to:
synthesize one or more one or more target nucleic acids in the plurality of target nucleic acids based at least in part on one or more or result subsequences in the plurality of result subsequences.

19. The apparatus of claim 17, wherein at least one of the one or more memories has further instructions stored thereon that, when executed by at least one of the one or more processors, cause at least one of the one or more processors to:
determine quantity of assembly reactions required to generate the plurality of result subsequences; and
divide the plurality of result subsequences into a plurality of assembly groups based at least in part on a determination that the quantity of assembly reactions required to generate the plurality of result subsequences is greater than a predetermined threshold.

20. The apparatus of claim 11, wherein at least one of the one or more memories has further instructions stored thereon that, when executed by at least one of the one or more processors, cause at least one of the one or more processors to:
filter the plurality of nucleic acid sequences to remove hairpin structures.

21. At least one non-transitory computer-readable medium storing computer-readable instructions for optimized partitioning for assembly of nucleic acid sequences that, when executed by one or more computing devices, cause at least one of the one or more computing devices to:
one or more processors; and
one or more memories operatively coupled to at least one of the one or more processors and having instructions stored thereon that, when executed by at least one of the one or more processors, cause at least one of the one or more processors to:
receive a plurality of nucleic acid sequences corresponding to a plurality of target nucleic acids for assembly and a plurality of synthesis parameters;
query an inventory database based at least in part on the plurality of nucleic acid sequences to determine a first plurality of matching nucleic acid subsequences, wherein the inventory database corresponds to a plurality of nucleic acid subsequences available in an inventory;
identify a second plurality of matching nucleic acid subsequences based at least in part on one or more overlaps between the plurality of nucleic acid sequences;
generate an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters; and
determine an optimal partitioning of the plurality of nucleic acid sequences based at least in part on an optimal path through the acyclic directed graph data structure that minimizes a total weight of traversed edges and nodes within the acyclic directed graph data structure.

22. The at least one non-transitory computer-readable medium of claim 21, wherein the instructions that, when executed by at least one of the one or more computing devices, cause at least one of the one or more computing devices to generate an acyclic directed graph data structure corresponding to potential partitions of the plurality of nucleic acid sequences based at least in part on the first plurality of matching nucleic acid subsequences, the second plurality of matching nucleic acid subsequences, and one or more synthesis parameters in the plurality of synthesis parameters further cause at least one of the one or more computing devices to:
determine a plurality of partition points of the plurality of nucleic acid sequences based at least in part on the first plurality of matching nucleic acid subsequences and the second plurality of matching nucleic acid subsequences;
generate one or more synthetic nodes representing one or more partition points in the plurality of partition points, each synthetic node corresponding to a partition adjacent to nucleic acid subsequences that are not in the first plurality of matching nucleic acid subsequences or the second plurality of matching nucleic acid subsequences;
generate one or more matching nodes representing one or more second partition points in the plurality of partition points, each matching node corresponding to a partition adjacent to at least one nucleic acid subsequence that is in the first plurality of matching nucleic acid subsequences or the second plurality of matching nucleic acid subsequences; and
generate a plurality of directed edges connecting the one or more synthetic nodes and the one or more matching nodes based at least in part on the one or more synthesis parameters in the plurality of synthesis parameters and the plurality of target nucleic acids.

23. The at least one non-transitory computer-readable medium of claim 22, wherein each edge in the plurality of directed edges has one or more associated edge properties, the one or more associated edge properties being determined based at least in part on one or more of:
whether the edge connects to a synthetic node or a matching node; or
the one or more synthesis parameters in the plurality of synthesis parameters.

24. The at least one non-transitory computer-readable medium of claim 21, wherein the plurality of synthesis parameters comprise one or more of a subsequence synthesis length range, an assembly strategy, one or more inventory partition parameters, a guanine or cytosine content overlap range, a subsequence overlap length, one or more overlap configuration parameters, one or more enzyme digestion parameters, a maximum match length parameter, or a maximum assembly subsequences per reaction.

25. The at least one non-transitory computer-readable medium of claim 21, wherein:
the first plurality of matching nucleic acid subsequences are determined based at least in part on one or more of the plurality of synthesis parameters; and
the second plurality of matching subsequences are determined based at least in part on one or more of the plurality of synthesis parameters.

26. The at least one non-transitory computer-readable medium of claim 21, wherein one or more nucleic acid sequences in the plurality of nucleic acid sequences comprise circular sequences and wherein first plurality of matching nucleic acid subsequences and the second plurality of matching subsequences are determined based at least in part on one or more rotations of the circular sequences.

27. The at least one non-transitory computer-readable medium of claim 21, further storing computer-readable instructions that, when executed by at least one of the one or more computing devices, cause at least one of the one or more computing devices to:
generate a plurality of result subsequences based at least in part on the optimal partitioning of the plurality of nucleic acid sequences, each result subsequence indicating a corresponding source, wherein one or more target nucleic acids in the plurality of target nucleic acids are configured to be sequenced based at least in part on one or more or result subsequences in the plurality of result subsequences.

28. The at least one non-transitory computer-readable medium of claim 27, further storing computer-readable instructions that, when executed by at least one of the one or more computing devices, cause at least one of the one or more computing devices to:
synthesize one or more one or more target nucleic acids in the plurality of target nucleic acids based at least in part on one or more or result subsequences in the plurality of result subsequences.

29. The at least one non-transitory computer-readable medium of claim 27, further storing computer-readable instructions that, when executed by at least one of the one or more computing devices, cause at least one of the one or more computing devices to:
determine quantity of assembly reactions required to generate the plurality of result subsequences; and
divide the plurality of result subsequences into a plurality of assembly groups based at least in part on a determination that the quantity of assembly reactions required to generate the plurality of result subsequences is greater than a predetermined threshold.

30. The at least one non-transitory computer-readable medium of claim 21, wherein at least one of the one or more memories has further instructions stored thereon that, when executed by at least one of the one or more processors, cause at least one of the one or more processors to:
filter the plurality of nucleic acid sequences to remove hairpin structures.
